(19) United States
(12) Patent Application Publication
(10) Pub. No.: US 2023/0076575 A1
(43) Pub. Date: Mar. 9, 2023

(54) MODEL PERSONALIZATION SYSTEM WITH OUT-OF-DISTRIBUTION EVENT DETECTION IN DIALYSIS MEDICAL RECORDS

(71) Applicant: NEC Laboratories America, Inc., Princeton, NJ (US)

(72) Inventors: Jingchao Ni, Princeton, NJ (US); Wei Cheng, Princeton Junction, NJ (US); Haifeng Chen, West Windsor, NJ (US)

(21) Appl. No.: 17/883,729

(22) Filed: Aug. 9, 2022

Related U.S. Application Data
(60) Provisional application No. 63/240,506, filed on Sep. 3, 2021.

(51) Int. Cl. G16H 50/30; G16H 50/70; G16H 50/50

(52) U.S. Cl. CPC G16H 50/30; G16H 50/70; G16H 50/50

(57) ABSTRACT

A method for making prognostic prediction scores during a pre-dialysis period on an incidence of events in future dialysis includes learning a meta-training model that simultaneously classifies dialysis in-distribution events and detects out-of-distribution (OOD) events during model personalization by employing a data preprocessing component to extract different parts of data from historical medical records of patients to generate a meta-training dataset, a meta-training component to analyze the meta-training dataset, the meta-training component including a class pool generator, a task generator, a prototype network, an attention component, and a model training component, the class pool generator splitting training classes into a first class pool and a second class pool for generating a distribution statistics dictionary, a storage component to store the meta-training model for distribution to local machines, and a personalization component including a local data collection component, and a class and OOD detector component.

### RELATED APPLICATION INFORMATION

This application claims priority to Provisional Application No. 63/240,506, filed on Sep. 3, 2021, the contents of which are incorporated herein by reference in their entirety.

### BACKGROUND

### Technical Field

The present invention relates to dialysis event prediction and, more particularly, to a model personalization system with out-of-distribution event detection in dialysis medical records.

### Description of the Related Art

Recently, the tremendous employments of digital systems in hospitals and medical institutions have brought forth a large volume of healthcare data of patients. The big data are of substantial value, which enables Artificial Intelligence (AI) to be exploited to support clinical judgement in medicine. As one of the critical themes in modern medicine, the number of patients with kidney diseases has raised social, medical and socioeconomic issues worldwide. Hemodialysis, or simply dialysis, is a process of purifying the blood of a patient whose kidneys are not working normally and is one of the important renal replacement therapies (RRT). However, dialysis patients at high risk of cardiovascular and other diseases require intensive management of blood pressure, anemia, mineral metabolism, and so on. Otherwise, patients may encounter critical events, such as low blood pressure, leg cramps, and even mortality, during dialysis. Therefore, medical staff must decide to start dialysis from various viewpoints. Some previous reports showed that variable clinical factors were related to dialysis events. As a result, given the availability of big medical data, it is beneficial to develop AI systems for making prognostic prediction scores during the pre-dialysis period on the incidence of events in future dialysis, which can largely facilitate the decision-making processes of medical staff, and hence reduce the risk of events.

### SUMMARY

A method for making prognostic prediction scores during a pre-dialysis period on an incidence of events in future dialysis is presented. The method includes learning a meta-training model that simultaneously classifies dialysis in-distribution events and detects out-of-distribution (OOD) events during model personalization by leveraging a data preprocessing component to extract different parts of data from historical medical records of patients to generate a meta-training dataset, a meta-training component to analyze the meta-training dataset, the meta-training component including a class pool generator, a task generator, a prototype network, an attention component, and a model training component, the class pool generator splitting training classes into a first class pool for generating training tasks and a second class pool for generating a distribution statistics dictionary for transfer learning, a storage component to store the meta-training model for distribution to local machines for further fine-tuning, personalization, and deployment, and a personalization component including a local data collection component, and a class and OOD detector component, the class and OOD detector component using an energy score and a pre-defined threshold for estimating out-of-distribution samples.

A non-transitory computer-readable storage medium comprising a computer-readable program for making prognostic prediction scores during a pre-dialysis period on an incidence of events in future dialysis is presented. The computer-readable program when executed on a computer causes the computer to perform the steps of learning a meta-training model that simultaneously classifies dialysis in-distribution events and detects out-of-distribution (OOD) events during model personalization by leveraging a data preprocessing component to extract different parts of data from historical medical records of patients to generate a meta-training dataset, a meta-training component to analyze the meta-training dataset, the meta-training component including a class pool generator, a task generator, a prototype network, an attention component, and a model training component, the class pool generator splitting training classes into a first class pool for generating training tasks and a second class pool for generating a distribution statistics dictionary for transfer learning, a storage component to store the meta-training model for distribution to local machines for further fine-tuning, personalization, and deployment, and a personalization component including a local data collection component, and a class and OOD detector component, the class and OOD detector component using an energy score and a pre-defined threshold for estimating out-of-distribution samples.

A system for making prognostic prediction scores during a pre-dialysis period on an incidence of events in future dialysis is presented. The system includes a data preprocessing component to extract different parts of data from historical medical records of patients to generate a meta-training dataset, a meta-training component to analyze the meta-training dataset, the meta-training component including a class pool generator, a task generator, a prototype network, an attention component, and a model training component, the class pool generator splitting training classes into a first class pool for generating training tasks and a second class pool for generating a distribution statistics dictionary for transfer learning, a storage component to store a meta-training model for distribution to local machines for further fine-tuning, personalization, and deployment, and a personalization component including a local data collection component, and a class and

OOD detector component, the class and OOD detector component using an energy score and a pre-defined threshold for estimating out-of-distribution samples. The data preprocessing component, the meta-training component, the storage component, and the personalization component are collectively used to learn the meta-training model that simultaneously classifies dialysis in-distribution events and detects out-of-distribution (OOD) events during model personalization.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIGS. 1A-1C illustrate a block/flow diagram of an exemplary framework for the Out-Of-Distribution (OOD) event detection problem, in accordance with embodiments of the present invention;

FIGS. 2A-2B illustrate a block/flow diagram of an exemplary architecture of the Out-of-distribution even Detection enhanced Model Personalization (ODMP) system, in accordance with embodiments of the present invention;

FIG. 3 is a block/flow diagram illustrating a sample generation of the preprocessing component, in accordance with embodiments of the present invention;

FIG. 4 is a block/flow diagram illustrating a prototype network structure, in accordance with embodiments of the present invention;

FIG. 5 is a block/flow diagram illustrating the workflow of the ODMP system, in accordance with embodiments of the present invention;

FIG. 6 is a block/flow diagram illustrating the functions of the ODMP meta-training component and the ODMP personalization component, in accordance with embodiments of the present invention;

FIG. 7 is a block/flow diagram illustrating the functions of the ODMP preprocessing component and the ODMP class pool generator, in accordance with embodiments of the present invention;

FIG. 8 is a block/flow diagram illustrating the functions of the ODMP task generator and the ODMP prototype network, in accordance with embodiments of the present invention;

FIG. 9 is a block/flow diagram illustrating the functions of the ODMP distribution dictionary and the ODMP attention component, in accordance with embodiments of the present invention;

FIG. 10 is a block/flow diagram illustrating the functions of the ODMP training component and the ODMP class and OOD detector, in accordance with embodiments of the present invention;

FIG. 11 is an exemplary practical application for making prognostic prediction scores during a pre-dialysis period on an incidence of events in future dialysis, in accordance with embodiments of the present invention;

FIG. 12 is an exemplary processing system for making prognostic prediction scores during a pre-dialysis period on an incidence of events in future dialysis, in accordance with embodiments of the present invention; and

FIG. 13 is a block/flow diagram of an exemplary method for making prognostic prediction scores during a pre-dialysis period on an incidence of events in future dialysis, in accordance with embodiments of the present invention.

### DETAILED DESCRIPTION OF PREFERRED EMBODIMENTS

Key challenges that prevent Artificial Intelligence (AI) systems from successfully being applied for precise analysis of medical data of patients include big variety and data limitation. Regarding big variety, due to the high variety of the population among patients, it is difficult for a single pre-trained model (trained on a set of historical patients' data) to be accurate for every new patient, who may have a different age, gender, genetics, health conditions, and so on. Regarding data limitation, because medical data usually includes sensitive information of patients, which raises privacy concerns during the data sharing process, it is difficult to obtain such data from hospitals at a sufficient scale for training an accurate and generalizable model.

Therefore, a single pre-trained model that is trained with such a limited training dataset is often not generalizable for predictive analysis on new patients' data. Specifically, unseen events that cannot be covered by the limited training data distribution are difficult to predict, and such events are thus named Out-Of-Distribution (OOD) data.

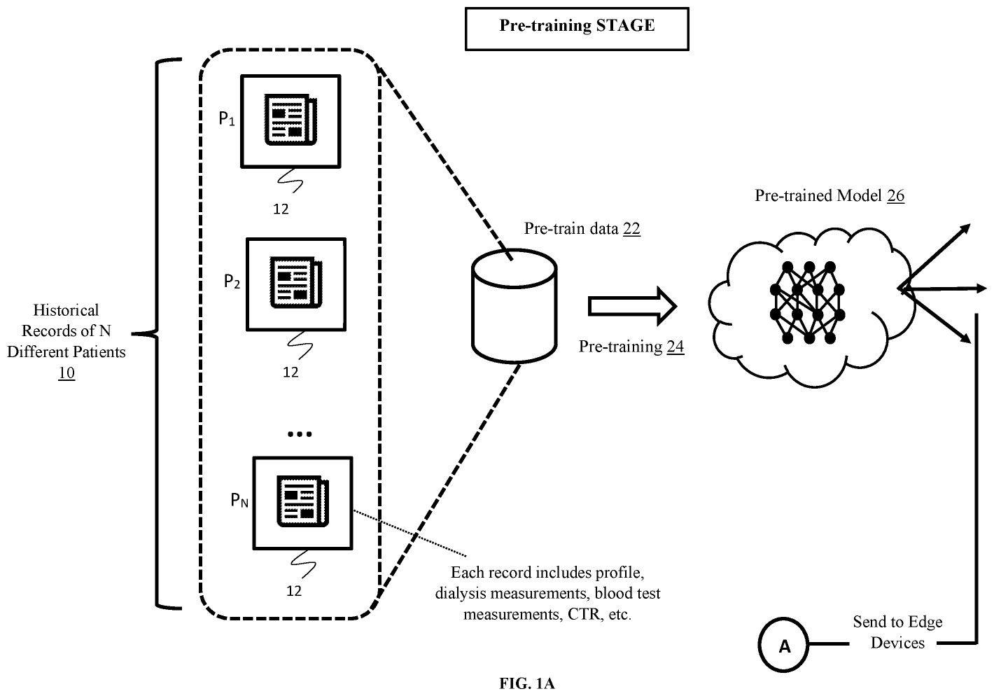

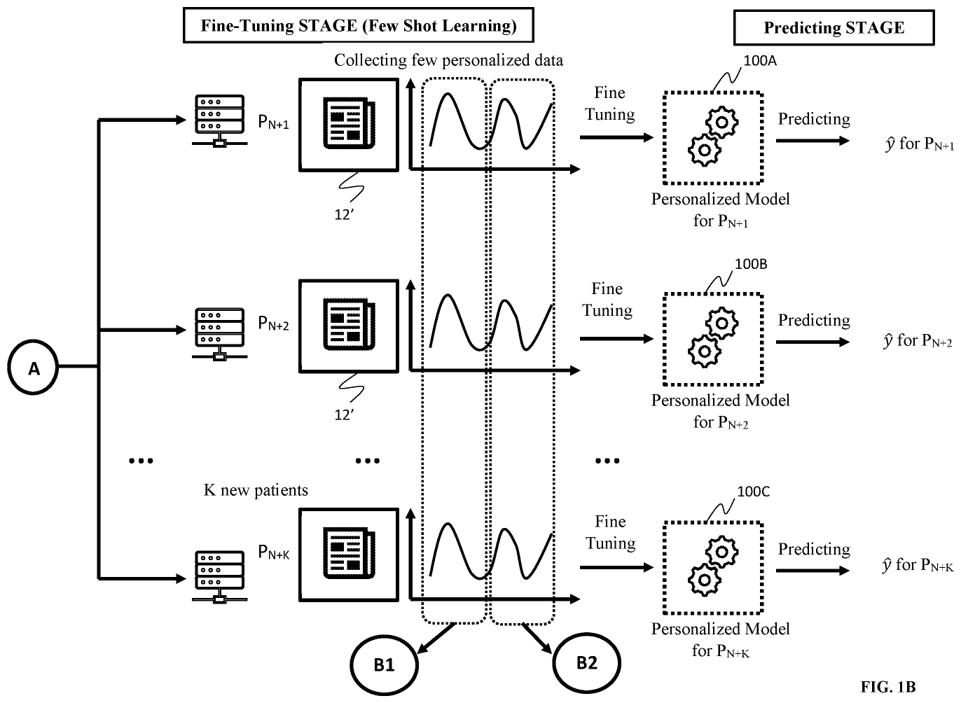

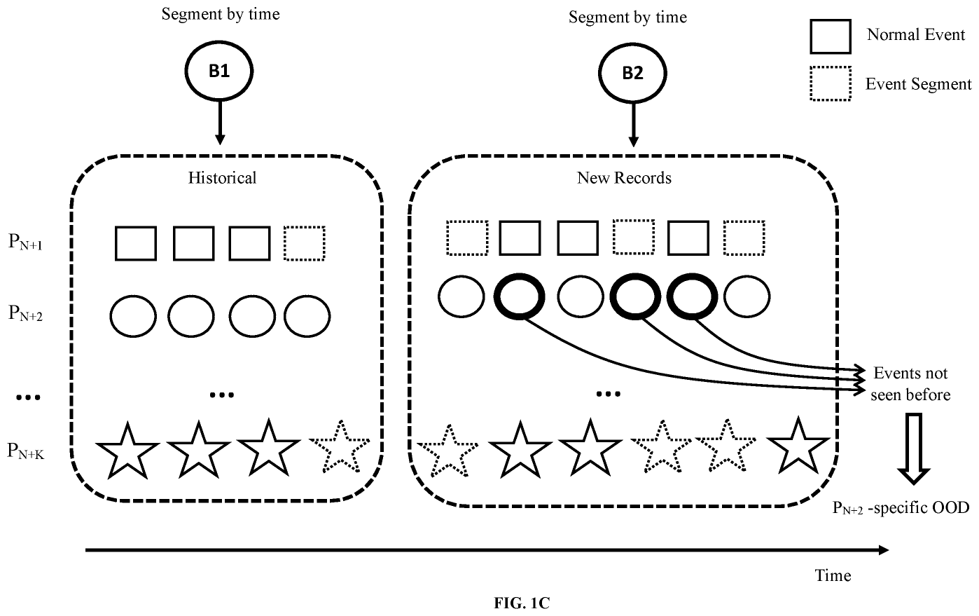

This present invention addresses the above-mentioned challenges by providing automatic and high-quality prognostic detection scores of OOD events. In particular, the present invention handles this problem under a model personalization framework, as illustrated by FIGS. 1A-1C.

Before delving into FIGS. 1A-1C, an introduction to the data description is presented. Specifically, dialysis patients have a regular routine of dialysis sessions with a frequency of 3 times per week. Each session takes about 4 to 5 hours to complete. The problem to solve is to predict the possibility of the incidence of events in a near future dialysis session for each patient based on the past recording data. The recording data of dialysis patients mainly include static profiles of the patients (e.g., age, gender, starting time of dialysis, etc.), dialysis measurement records (with a frequency of 3 times/week, e.g., blood pressure, weight, venous pressure, etc.), blood test measurements (with a frequency of 2 times/month, e.g., albumin, glucose, platelet count, etc.), and cardiothoracic ratio (CTR, with a frequency of 1 time/month). The last three are dynamic and change over time, so they can be modeled by a time series, but with different frequencies.

The model personalization framework aims to leverage a small amount of a patient's data to personalize a pretrained model so that the personalized model generalizes better to the new data distribution and provides more accurate prediction for that patient. The framework has the following exemplary stages:

A pretraining stage (FIG. 1A) that uses the available historical data 10 of patients P₁ to P_{N} (12) to pretrain 24 an initial model 26 with pre-trained data 22, which is stored on the cloud platform for future use. Because the historical data is limited, the initial model 26 may not be generalizable to different new patient data.

A finetuning stage (FIGS. 1B, 1C) that collects a short period of new records data 12′ for every new patient, P_{N+1} to P_{N+K}, then the pretrained model is sent to the edge devices where P_{N+1} to P_{N+K} are located. The finetuning stage uses this small amount of newly collected data to finetune the pretrained model, and finally each edge device has a personalized model, which may be different from each other.

A predicting stage (FIG. 1B) that uses the personalized models 100A, 110B, 100C after finetuning for prediction, which is better than directly using the original pre-trained model.

During the second stage, because it is often likely that events do not happen during the short new data collection period, it is possible that the finetuning processes of some patient tasks are unaware of events distribution, such as P_{N+2}. As such, when there are new events in the testing time, they are unseen events to the personalized model, and are difficult to predict because they are Out-Of-Distribution (OOD).

The present invention addresses this problem by leveraging the techniques of meta-learning and OOD detection and is carefully devised to have a meta-pre-training strategy for learning a model that simultaneously classifies in-distribution events and detects OOD events. Meanwhile, the meta-pre-training strategy fits quick finetuning with a small or limited amount of data and performs well in the personalized domain. The present invention thus provides for a meta-training model that can do both classification (in-distribution event prediction) and OOD detection (out-of-distribution event prediction) in the model personalization scenario. Thus, the present invention is named Out-of-distribution event Detection enhanced Model Personalization (ODMP) system.

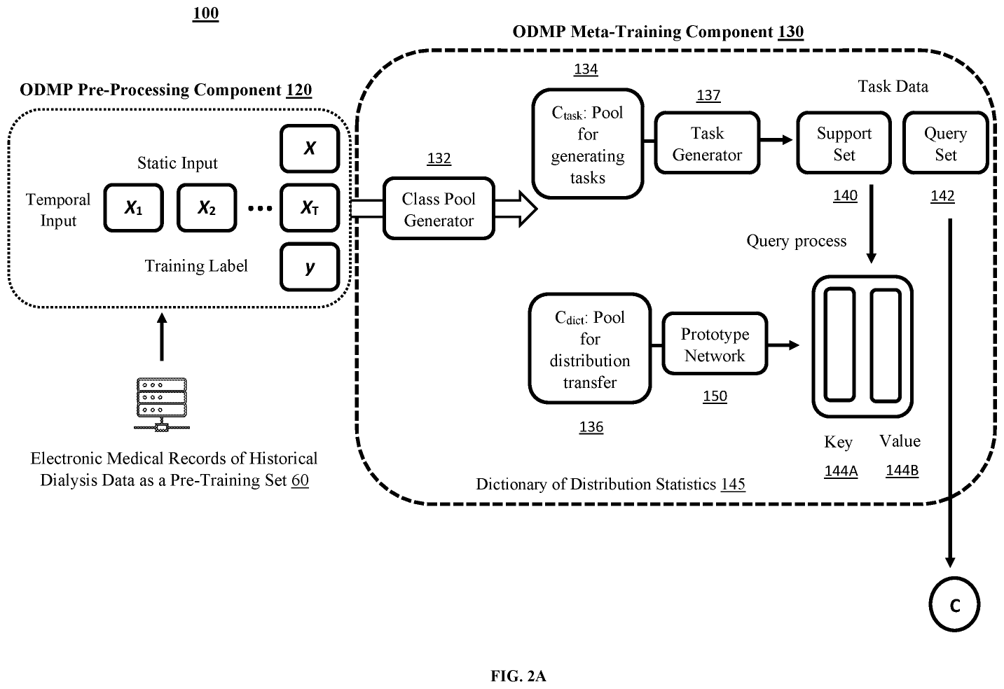

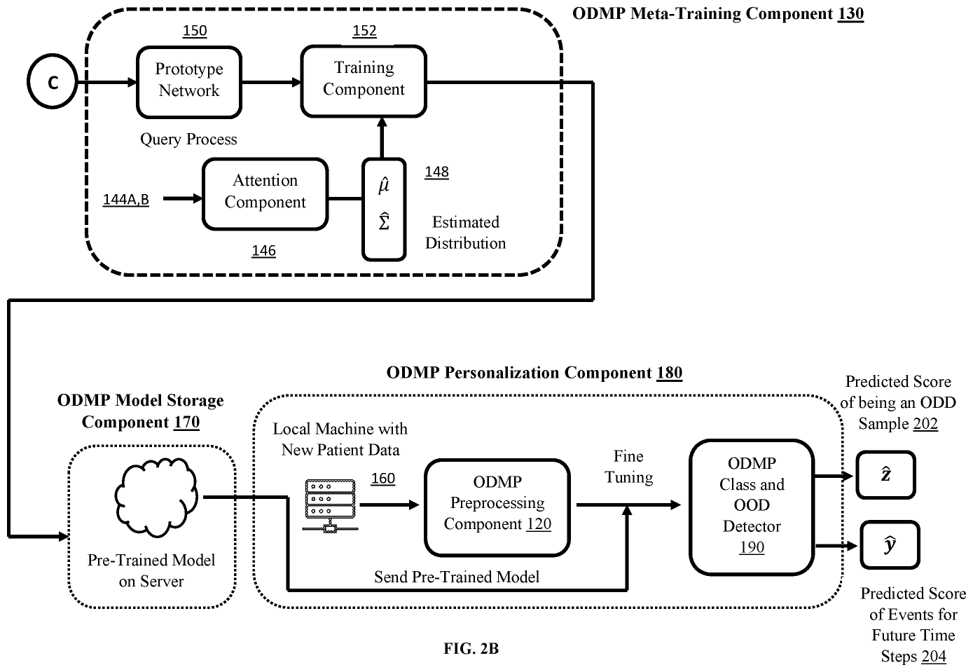

FIGS. 2A-2B show the overall architecture of the ODMP system 100. The components include a ODMP data preprocessing component 120, a ODMP meta-training component 130, a ODMP model storage component 170, and a ODMP model personalization component 180.

Regarding the ODMP data preprocessing component 120, the historical records of dialysis patients can be stored in forms such as cvs and excel files. Each patient has a file that includes information on static profile, dialysis measurements, blood test measurements, and event incidences. Each row indicates a particular date of a hospital visit by the patient. Each column indicates a particular feature, such as some indicator metrics in the dialysis measurements (e.g., blood pressure, weight, venous pressure, etc.). Since different parts have different frequencies, some entries in the form can be blank indicating that feature is not measured at a particular date.

The data preprocessing component 120 extracts different parts of the data from the files, removes noisy information, and fills in some missing values by using mean values of the corresponding features in the historical data or by using values from adjacent earlier time steps.

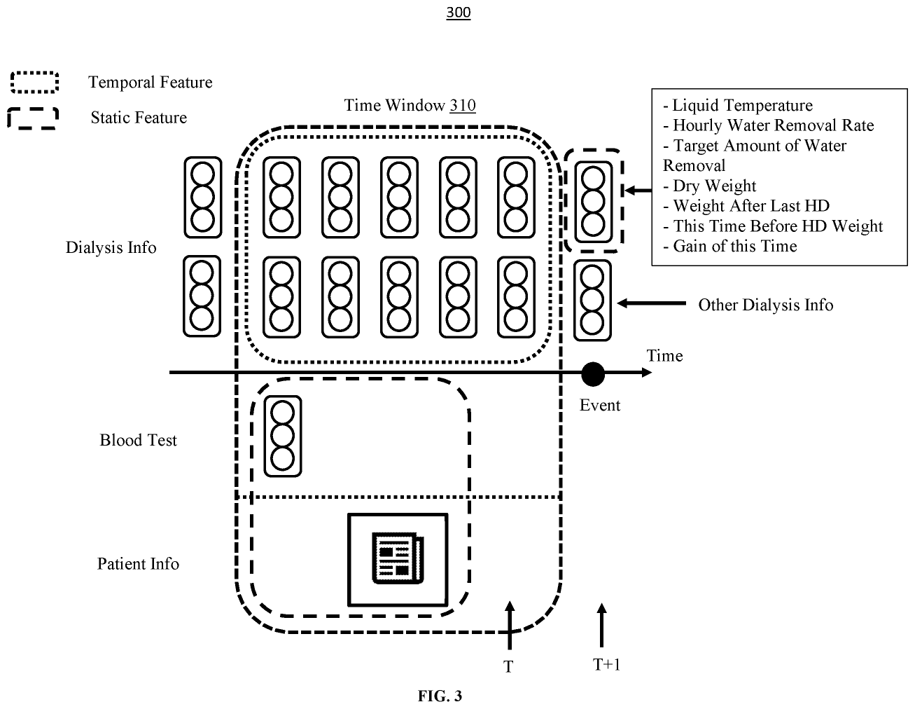

Moreover, the data preprocessing component 120 sets up a time window of width w to segment the time series data. FIG. 3 illustrates the segmentation process 300. Each time window 310 generates a sample X from time step T−w to time step T, and associates it with an event label Y at time step T+1. The purpose is to generate samples that focus on the features in the closest dates to a future event. Because different parts have different frequencies, all dialysis measurements in the time window will be included, while the blood test measurements on the closest date to the time window will be included. Then the time window will slide from the beginning of the date to the end of the date in the records to generate multiple samples.

In particular, some of the dialysis measurements are evaluated on the same date for which the event is to be predicted. These measurements are evaluated immediately before the dialysis starts. Thus, they can be included as static features as illustrated by the boxed features on the upper right corner of FIG. 3.

After samples are generated, the data preprocessing component 120 will normalize all the samples by using a Gaussian normalization method such that the features of the training samples have a mean of 0 and a variance of 1, which facilitates the stability of the computing algorithm in the next steps. For testing samples, they are normalized by using the mean and variance obtained from the training data. Then, the normalized samples are sent to the next component for model training and testing.

Regarding the ODMP meta-training component 130, it includes the following components: a class pool generator 132, a task generator 137, a prototype network 150, an attention component 146, and a training component 152.

Regarding the class pool generator 132, for multi-class classification tasks, the class pool generator 132 splits the training classes into two parts, that is, Cₜₐₛₖ 134 for generating training tasks, e.g., generating a support set 140 and a query set 142, and C_{dict} 136 for generating distribution statistics for transfer learning.

The Cₜₐₛₖ pool 134 is used to generate the support set 140 by only selecting classes that represent in-distribution data. Meanwhile, Cₜₐₛₖ pool 134 is also used to generate the query set 142 by selecting both in-distribution classes and several other classes to represent out-of-distribution data.

The C_{dict} pool 136 is designed to address the challenge of using limited data for estimating in-distribution. Usually, the support set 140 only has limited data, which cannot provide accurate distribution estimation. The intuition here is to leverage class similarity for improving the distribution estimation accuracy.

The C_{dict} pool data 136 are used to construct a distribution statistics dictionary 145, as illustrated in FIGS. 2A-2B. This dictionary 145 includes the mean and covariance (148) of every class in the C_{dict} pool 136. The dictionary 145 is stored as a memory for a querying step by using the mean of the classes in the support set 140.

Regarding the task generator 137, it is noted that the ODMP meta-training component 130 considers each of the patient's data as a task. The model is pre-trained iteratively from task to task so that the knowledge shared by different tasks can be extracted, and quickly adapted to new tasks. This is similar in a manner that humans quickly learn to deal with a new task by leveraging the knowledge learned from other relevant tasks.

The task generator 137 is responsible for organizing the patients' data in the training set into the format of tasks. Each task includes two subsets of data of one patient, the support set 140 and the query set 142. As such, it is supposed that there are N patients in the training set, N tasks are constructed, where every task has a support set and query set for the meta-training algorithm to coordinate.

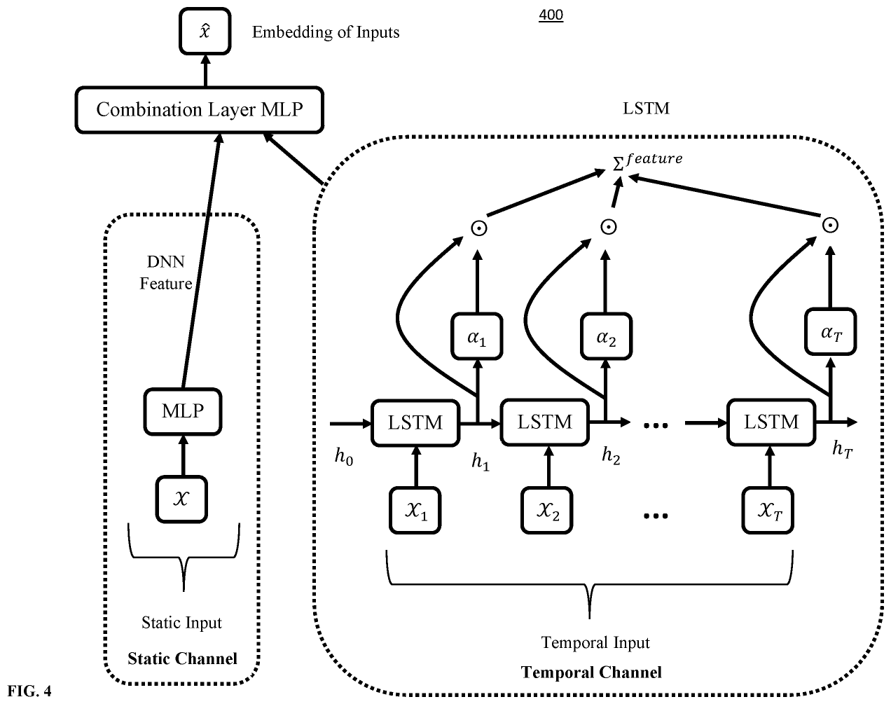

Regarding the prototype network 150, the prototype network 150 is responsible for encoding input data into feature vectors. Because the input data include both static information and time series information, a Dual-Channel Combination Network (DCCN) 400 is employed as the prototype network, which is illustrated in FIG. 4.

The prototype network 150 includes two channels, a static channel for processing static and low frequency temporal features, and a temporal channel for processing high frequency temporal features. Suppose the static features (and low frequency temporal features) are represented by a vector xₛ, the static channel has a Multilayer Perceptron (MLP) to encode the information in xₛ to a compact representation hₛ by:

hₛ=f_{MLP}(xₛ)

where f_{MLP}(⋅) can be multiple layers of a fully connected network with form Wₛxₛ+bₛ, with Wₛ and bₛ as model parameters to be trained.

After this step, the output hₛ will be a compact representation of the static features, which will be integrated with the representations from temporal channels for prediction.

The temporal channel includes several Long Short-Term Memory (LSTM) layers for processing the temporal features. Suppose the temporal features are represented by a sequence of vectors x₁, . . . , x_{T}, the LSTM layers will output a sequence of compact representations h₁, . . . , h_{T} by: h₁, . . . , h_{T}=f_{LSTM}(x₁, . . . , x_{T}),

where f_{LSTM}(⋅) can have multiple layers of LSTM units, which include trainable model parameters. Also, the LSTM units can be extended to a bi-directional LSTM to encode information from both temporal directions.

On top of the LSTM layers, h₁, . . . , h_{T} will be sent to an attention layer for combination. The attention layer calculates a temporal importance score, i.e., attention weight αₜ, for each time step by:

eₜ=w_{α} tan h(W_{α}hₜ) for t=1, . . . ,T

αₜ=softmax(eₜ) for t=1, . . . ,T

where W_{α} and w_{α} are model parameters to learn. After this step, Σₜ₌₁^{T}αₜ=1.

Then, all compact temporal representations will be combined through the attention weights by:

h_{d}=Σₜ₌₁^{T}αₜhₜ

where h_{d} is a compact representation for all temporal features x₁, . . . , x_{T}.

After the static and temporal representations hₛ and h_{d} are obtained from the static channel and temporal channel, the combination layer concatenates them and computes the embedding vector:

\hat{x}=f_{MLP}([hₛ,h_{d}])

where \hat{x} is a feature vector which encodes the input information.

Regarding the attention component 146, the attention component 146 is used for the query step, which receives the mean of a support set class as input and outputs the transferred distribution statistics, including a calibrated mean and a transferred covariance.

The attention component 146 has an MLP for computing the attention score as follows:

$${a}_{j} = \frac{exp [ sim ( {g}_{φ} ( {μ}_{s} ) , {g}_{φ} ( {μ}_{j} ) ) / τ ]}{{\sum }_{i = 1}^{\vert {C}_{dict} \vert} exp [ sim ( {g}_{φ} ( {μ}_{s} ) , {g}_{φ} ( {μ}_{i} ) ) / τ ]}$$

where μₛ is the mean of a support set class, μⱼ (j=1, . . . , |C_{dict}|) is the mean of the j-th class in the C_{dict} pool. 136 The sim( ) function is a similarity function, where the exemplary methods use negative Euclidean distance or cosine similarity for realizing this function. τ is a hyperparameter that represents temperature. The output aⱼ is an attention score that represents how similar the input support set class to the j-th class in the C_{dict} pool 136.

After obtaining the attention scores aⱼ for j=1, . . . , |C_{dict}|, the attention component 146 computes a calibrated mean as:

$${\hat{μ}}_{s} = \frac{{\sum }_{i = 1}^{C} {a}_{i} {μ}_{i} + {μ}_{s}}{2}$$

and computes a transferred covariance as:

\hat{Σ}= [symbol] ′_{ω}([\hat{Σ}₁, . . . ,\hat{Σ}_{N},\hat{μ}₁, . . . ,\hat{μ}_{N}])

where \hat{Σ}_{y}=Σᵢ₌₁^{c}aᵢΣᵢ+α (y=1, . . . , N), and [symbol] ′_{ω}( ) is a function realized by an MLP.

Regarding the training component 152, the training component 152 receives inputs from both the support set 140 and the query set 142 generated by the task generator 137.

The loss function includes two parts:

[symbol]

= [symbol] _{CL}+λ [symbol] _{EN}

where the first part is a cross-entropy loss for classifying whether a segment sample is a normal segment or event, and the second part is an energy-based model for detecting OOD events.

Specifically, the loss function can be written as:

$$ℓ = {\underbrace{- \frac{1}{{n}_{in}} {\sum }_{( x , y ) ∈ {𝒟}_{in}} log p ( y \vert x )}}_{ℓ OL} - λ {\underbrace{\frac{1}{{n}_{in}} {\sum }_{x ∈ {𝒟}_{in}} [ log p ( x ) - \frac{1}{r} {\sum }_{{x}_{i}^{′} ∼ {𝒟}_{out} , i = 1}^{r} log p ( {x}_{i}^{′} ) ]}}_{{ℓ}_{EN}}$$

$$where$$

$$p ( y \vert x ) = \frac{exp ( - d ( x , {\hat{μ}}_{y} ) / τ )}{{\sum }_{{y}^{′} = 1}^{C} exp ( - d ( x , {\hat{μ}}_{{y}^{′}} ) / τ )}$$

log p(x)=−E(x)/τ−log Z

$$E ( x ) = - τ log {\sum }_{{y}^{′} = 1}^{c} exp ( - d ( x , {\hat{μ}}_{{y}^{′}} ) / τ )$$

$$d ( x , {\hat{μ}}_{y} ) = \frac{1}{2} {( x - {\hat{μ}}_{y} )}^{⊤} {\hat{Σ}}^{- 1} ( x - {\hat{μ}}_{y} )$$

and the distance function d( ) receives the outputs of the attention component 146, that is, the mean and covariance (148), and the model parameters are included in this distance function.

Meanwhile, the training component 152 has an adversarial sample enhanced training algorithm, which adds some adversarial noises to the OOD samples in the query set 142 for shrinking the in-distribution boundaries, thus facilitating better detection of the OOD events. Its sampling process can be summarized by:

Sample x′ᵢ from [symbol] ₒᵤₜ.

Add a small perturbation: \hat{x}′ᵢ=x′ᵢ+ε sign(∇ₓ log(p(x)).

Calculate the loss function [symbol] .

Regarding the ODMP model storage component 170, after the ODMP model is meta-trained through the meta-training component 130, it (together with all parameters updated and fixed) is sent to a server or a cloud platform for storage, so that it can be easily distributed to local machines for further finetuning and personalization using a small or limited number of records from new patients that are collected by the local machines.

Regarding the ODMP personalization component 180, in practice, when a new patient has performed dialysis for several weeks, the local machine collects several records for that patient during the time. Although the number of records is much smaller than the data size in the pre-training dataset, these records are specific to the particular patient and are valuable to adapt the globally pre-trained model to the contexts of the particular patient. This personalization process via a small amount of finetuned data leverages the advantages of few-shot learning. ODMP is meta-trained specifically for leveraging a small or limited amount of data for quick adaptation. The following steps are conducted in component 180:

The meta-trained ODMP is sent to the local machine 160 where the finetuned dataset is collected and stored. The finetuned dataset is sent to the ODMP preprocessing component 120 for generating training samples in the support set 140. The meta-trained ODMP component 130 uses the prototype network 150, the dictionary 145, and the attention component 146 to estimate the mean and variance (148) of the new support set.

With the estimated mean and variance (148), the ODMP component 180 performs OOD detection by computing the energy score E(x) and uses a pre-defined threshold to determine OOD samples as:

$$F ( x ) = \{ \begin{matrix}1 , & E ( x ) > t \\ 0 , & E ( x ) \leq t\end{matrix}$$

Then for those regarded as in-distribution samples, the ODMP system 100 computes the classification probability as the predictive score of events, which is as follows

$$p ( y \vert x ) = \frac{exp ( - d ( x , {\hat{μ}}_{y} ) / τ )}{{\sum }_{{y}^{′} = 1}^{C} exp ( - d ( x , {\hat{μ}}_{{y}^{′}} ) / τ )}$$

Through this two-step approach, the ODMP system 100 simultaneously detects in-distribution and out-of-distribution events. Its meta-training design makes it suitable for quick adaptation with few samples. Predictions obtained in this manner are often significantly better than a model without pre-training or using the pre-trained model directly.

In conclusion, for the meta-training ODMP, the historical recording data 60 of dialysis patients are input to the ODMP data preprocessing component 120 and normalized samples are output as the meta-training set. Then the normalized samples are sent to the ODMP meta-training component 130, which includes a class pool generator 132, a task generator 137, feature embedding by a prototype network 150, query and distribution estimation through an attention component 146, and a model training component 152. Further, the meta-trained ODMP is sent to the model storage component 170 for future deployment and personalization in local machines.

For the fine-tuning ODMP and model testing, the small or limited amount of collected data is input in a local machine via the ODMP data preprocessing component 120 and normalized samples are output as the finetuning set. Then the meta-trained ODMP is sent from the model storage component 170 to the ODMP personalization component 180. Through the prototype network 150, the dictionary 145, and the attention component 146, the mean and variance (148) of the support set 140 are estimated. Thus, out-of-distribution events and classifying in-distribution samples are detected by using the two-step approach in the ODMP class and OOD detector component 190. This prediction is personalized because it uses the personal data from the local machines 160. The output includes predicted scores 202 of being OOD samples and personalized predicted scores 204 of events for future time steps.

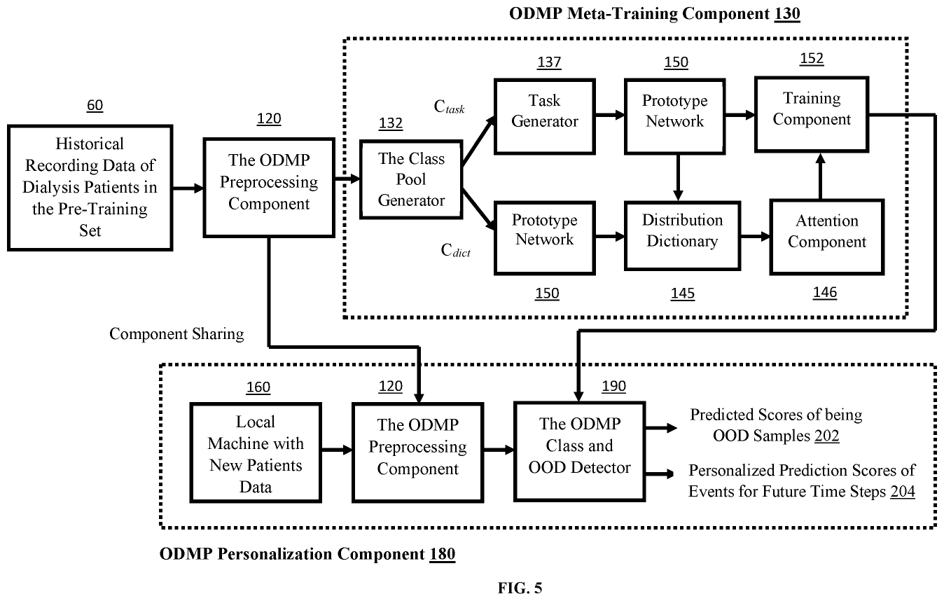

FIG. 5 is a block/flow diagram illustrating the workflow of the ODMP system, in accordance with embodiments of the present invention.

Historical recording data 60 of dialysis patients is fed into the ODMP preprocessing component 120. The data is then fed into the ODMP meta-training component 130, where a class pool generator 137 splits the training classes into Cₜₐₛₖ and Cₚₒₒₗ. The ODMP meta-training component 130 includes a prototype network 150, an attention component 146, and a training component 152. A distribution dictionary 145 is also provided. The data is then provided to a ODMP personalization component 180 that includes local machines 160 with new patient data and the ODMP class and OOD detector 190. The output includes predicted scores 202 of being OOD samples and personalized predicted scores 204 of events for future time steps.

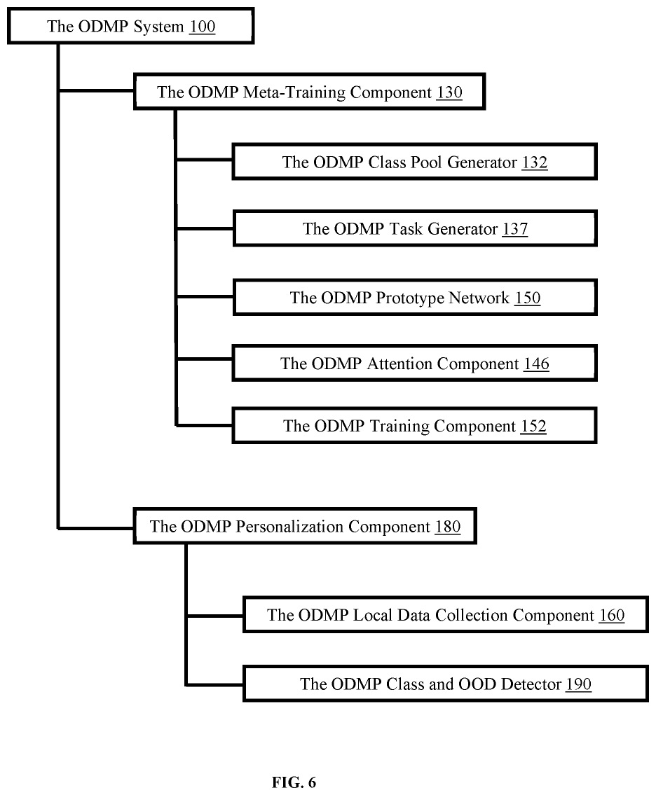

FIG. 6 is a block/flow diagram illustrating the functions of the ODMP meta-training component and the ODMP personalization component, in accordance with embodiments of the present invention.

The ODMP system 100 includes at least a ODMP meta-training component 130 and a ODMP personalization component 180.

The ODMP meta-training component 130 includes a ODMP class pool generator 132, a ODMP task generator 137, a ODMP prototype network 150, a ODMP attention component 146, and a ODMP training component 152.

The ODMP personalization component 180 includes a ODMP local data collection component 160 and a ODMP class and OOD detector 190.

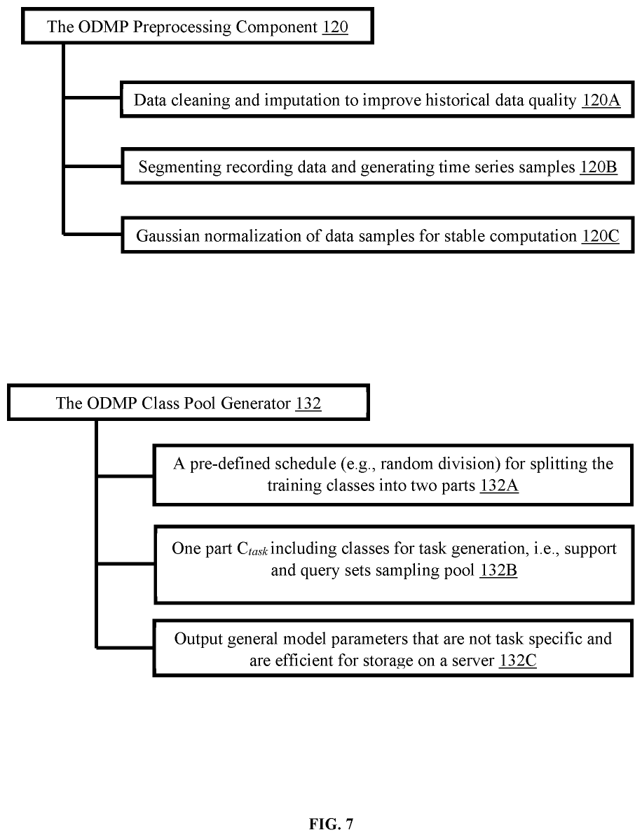

FIG. 7 is a block/flow diagram illustrating the functions of the ODMP preprocessing component and the ODMP class pool generator, in accordance with embodiments of the present invention.

The ODMP preprocessing component 120 includes the functions of:

Data cleaning and imputation to improve historical data quality 120A.

Segmenting recording data and generating time series samples 120B.

Gaussian normalization of data samples for stable computation 120C.

The ODMP class pool generator 132 includes:

A pre-defined schedule (e.g., random division) for splitting the training classes into two parts 132A.

One part, Cₜₐₛₖ, including classes for task generation, i.e., support and query sets sampling pool 132B.

Output general model parameters that are not task specific and are efficient for storage on a server 132C.

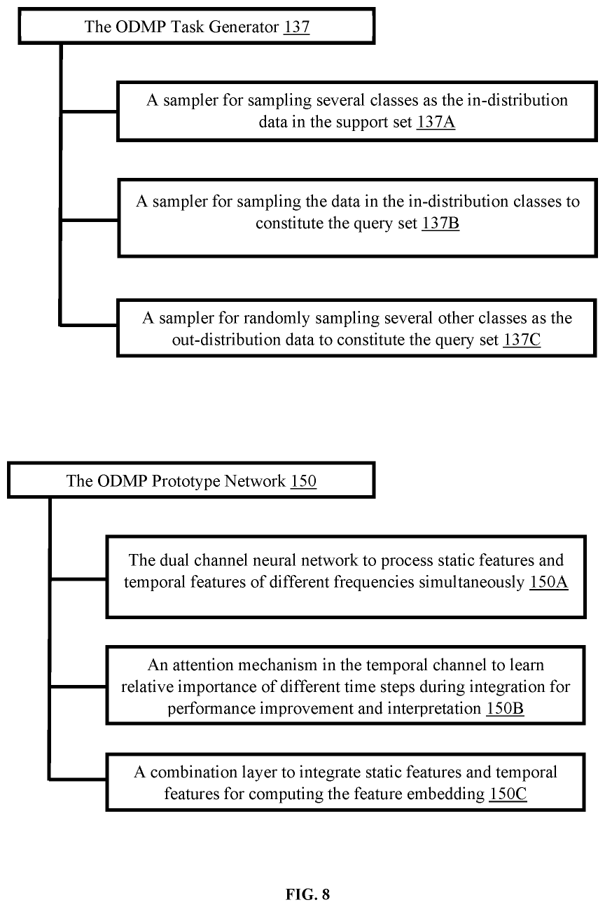

FIG. 8 is a block/flow diagram illustrating the functions of the ODMP task generator and the ODMP prototype network, in accordance with embodiments of the present invention.

The ODMP task generator 137 includes:

A sampler for sampling several classes as the in-distribution data in the support set 137A.

A sampler for sampling the data in the in-distribution classes to constitute the query set 137B.

A sampler for randomly sampling several other classes as the out-distribution data to constitute the query set 137C.

The ODMP prototype network 150 includes:

The dual channel neural network to process static features and temporal features of different frequencies simultaneously 150A.

An attention mechanism in the temporal channel to learn relative importance of different time steps during integration for performance improvement and interpretation 150B.

A combination layer to integrate static features and temporal features for computing the feature embedding 150C.

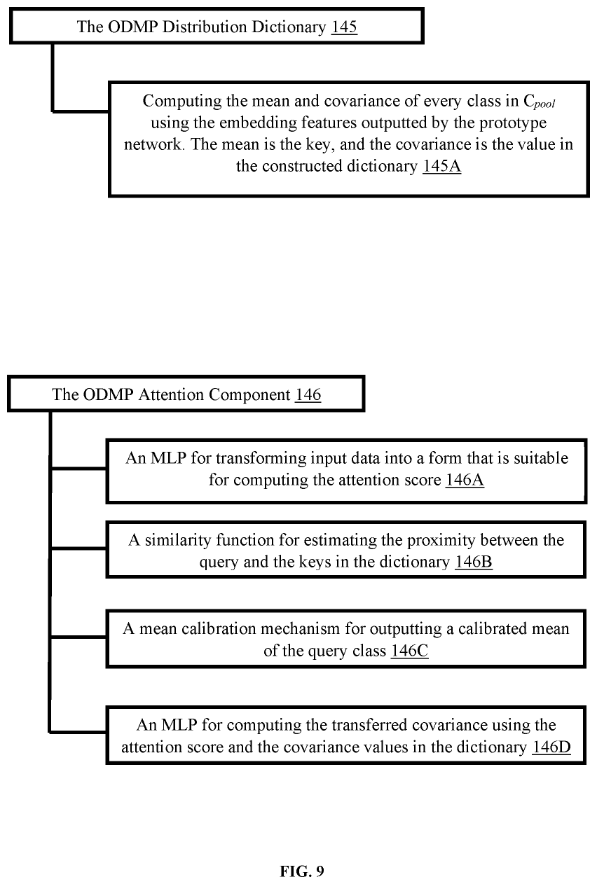

FIG. 9 is a block/flow diagram illustrating the functions of the ODMP distribution dictionary and the ODMP attention component, in accordance with embodiments of the present invention.

The ODMP distribution dictionary 145 includes computing the mean and covariance of every class in Cₚₒₒₗ using the embedding features outputted by the prototype network. The mean is the key (144A), and the covariance is the value (144B) in the constructed dictionary 145A.

The ODMP attention component 146 includes:

An MLP for transforming input data into a form that is suitable for computing the attention score 146A.

A similarity function for estimating the proximity between the query and the keys in the dictionary 146B.

A mean calibration mechanism for outputting a calibrated mean of the query class 146C.

An MLP for computing the transferred covariance using the attention score and the covariance values in the dictionary 146D.

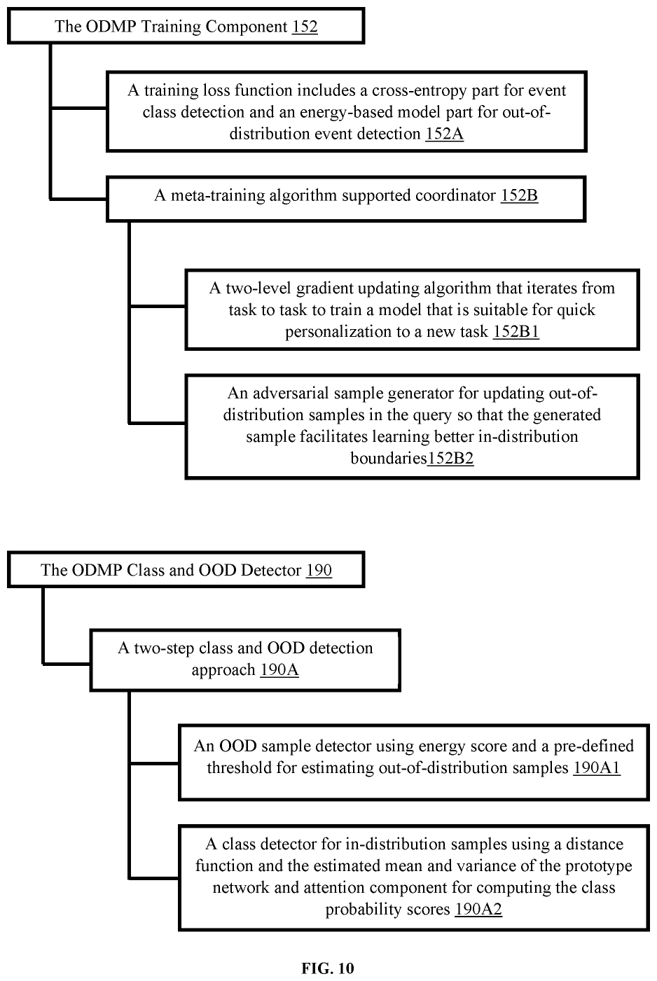

FIG. 10 is a block/flow diagram illustrating the functions of the ODMP training component and the ODMP class and OOD detector, in accordance with embodiments of the present invention.

The ODMP training component 152 includes:

A training loss function includes a cross-entropy part for event class detection and an energy-based model part for out-of-distribution event detection 152A.

A meta-training algorithm supported coordinator 152B, which further includes:

A two-level gradient updating algorithm that iterates from task to task to train a model that is suitable for quick personalization to a new task 152B1.

An adversarial sample generator for updating out-of-distribution samples in the query so that the generated sample facilitates learning better in-distribution boundaries 152B2.

The ODMP class and OOD detector 190 includes:

A two-step class and OOD detection approach 190A including:

An OOD sample detector using energy score and a pre-defined threshold for estimating out-of-distribution samples 190A1.

A class detector for in-distribution samples using a distance function and the estimated mean and variance of the prototype network and attention component for computing the class probability scores 190A2.

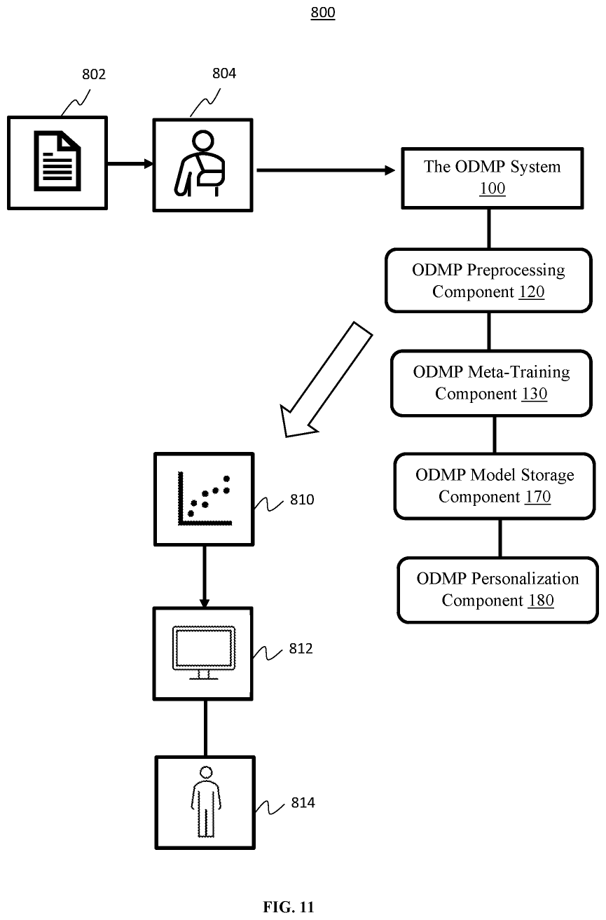

FIG. 11 is a block/flow diagram 800 of a practical application for making prognostic prediction scores during a pre-dialysis period on an incidence of events in future dialysis, in accordance with embodiments of the present invention.

In one practical example, records 802 of patients 804 are processed by the ODMP system 100 via a ODMP preprocessing component 120, a ODMP meta-training component 130, a ODMP model storage component 170, and a ODMP personalization component 180. The results 810 (e.g., variables or parameters or factors or features or records or medical data) can be provided or displayed on a user interface 812 handled by a user 814.

Therefore, a systematic and big data driven solution is provided to the problem of dialysis in-distribution event and out-of-distribution event prediction during model personalization.

ODMP system 100 is a neural network based intelligent computing system that does not require much human efforts on feature engineering.

ODMP system's 100 data encoding component, meta-training component 130, and personalization component 180 are designed specifically as an intelligent system for processing dialysis recording data.

ODMP system 100 formulates tasks from historical data for meta-training and has a meta-training strategy that trains the model to have better generalization capability to new data distributions.

ODMP system 100 has a meta-training strategy that trains the model to have the capability to detect both in-distribution and out-of-distribution events. A model that is such trained can quickly fit a new task with a small or limited amount of data, and perform well in the personalized domain.

ODMP system 100 addresses and alleviates the challenges of insufficient training data, and the distribution discrepancy of patients' data, and is thus promising to provide better accuracy than models without personalization or without consideration of OOD events.

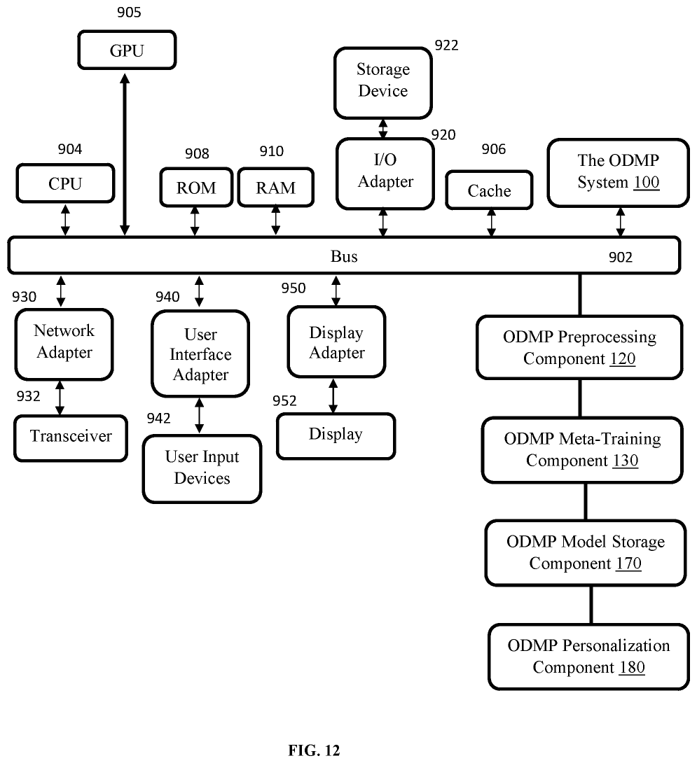

FIG. 12 is an exemplary processing system for making prognostic prediction scores during a pre-dialysis period on an incidence of events in future dialysis, in accordance with embodiments of the present invention.

The processing system includes at least one processor (CPU) 904 operatively coupled to other components via a system bus 902. A GPU 905, a cache 906, a Read Only Memory (ROM) 908, a Random Access Memory (RAM) 910, an input/output (I/O) adapter 920, a network adapter 930, a user interface adapter 940, and a display adapter 950, are operatively coupled to the system bus 902. Additionally, the ODMP system 100 includes a ODMP preprocessing component 120, a ODMP meta-training component 130, a ODMP model storage component 170, and a ODMP personalization component 180.

A storage device 922 is operatively coupled to system bus 902 by the I/O adapter 920. The storage device 922 can be any of a disk storage device (e.g., a magnetic or optical disk storage device), a solid-state magnetic device, and so forth.

A transceiver 932 is operatively coupled to system bus 902 by network adapter 930.

User input devices 942 are operatively coupled to system bus 902 by user interface adapter 940. The user input devices 942 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present invention. The user input devices 942 can be the same type of user input device or different types of user input devices. The user input devices 942 are used to input and output information to and from the processing system.

A display device 952 is operatively coupled to system bus 902 by display adapter 950.

Of course, the processing system may also include other elements (not shown), as readily contemplated by one of skill in the art, as well as omit certain elements. For example, various other input devices and/or output devices can be included in the system, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system are readily contemplated by one of ordinary skill in the art given the teachings of the present invention provided herein.

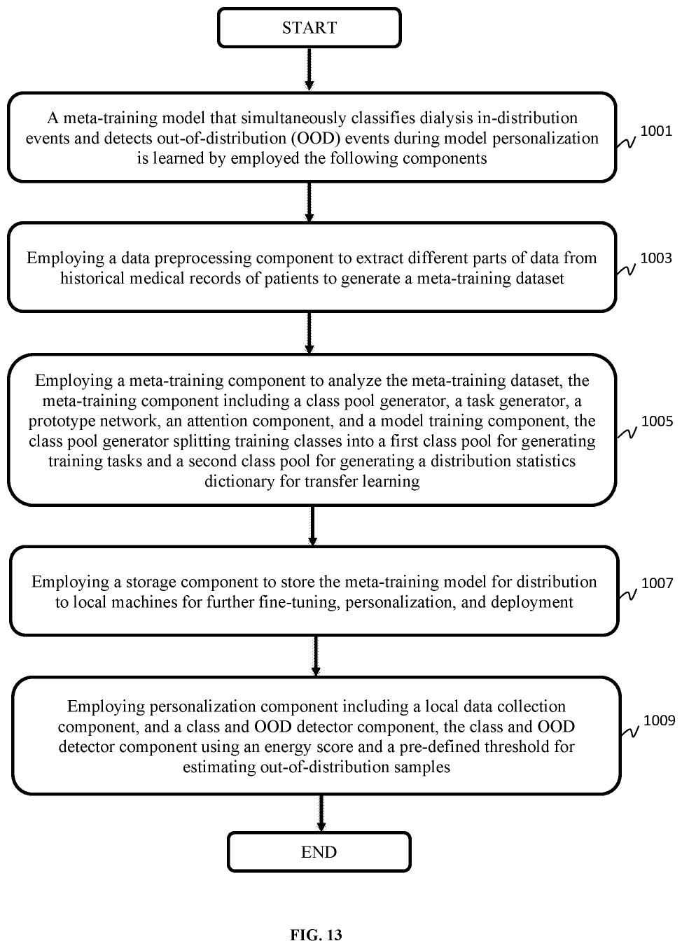

FIG. 13 is a block/flow diagram of an exemplary method for making prognostic prediction scores during a pre-dialysis period on an incidence of events in future dialysis, in accordance with embodiments of the present invention.

At block 1001, a meta-training model that simultaneously classifies dialysis in-distribution events and detects out-of-distribution (OOD) events during model personalization is learned by employing the following components:

At block 1003, a data preprocessing component is employed to extract different parts of data from historical medical records of patients to generate a meta-training dataset.

At block 1005, a meta-training component is employed to analyze the meta-training dataset, the meta-training component including a class pool generator, a task generator, a prototype network, an attention component, and a model training component, the class pool generator splitting training classes into a first class pool for generating training tasks and a second class pool for generating a distribution statistics dictionary for transfer learning.

At block 1007, a storage component is employed to store the meta-training model for distribution to local machines for further fine-tuning, personalization, and deployment.

At block 1009, a personalization component is employed including a local data collection component, and a class and OOD detector component, the class and OOD detector component using an energy score and a pre-defined threshold for estimating out-of-distribution samples.

As used herein, the terms “data,” “content,” “information” and similar terms can be used interchangeably to refer to data capable of being captured, transmitted, received, displayed and/or stored in accordance with various example embodiments. Thus, use of any such terms should not be taken to limit the spirit and scope of the disclosure. Further, where a computing device is described herein to receive data from another computing device, the data can be received directly from the another computing device or can be received indirectly via one or more intermediary computing devices, such as, for example, one or more servers, relays, routers, network access points, base stations, and/or the like. Similarly, where a computing device is described herein to send data to another computing device, the data can be sent directly to the another computing device or can be sent indirectly via one or more intermediary computing devices, such as, for example, one or more servers, relays, routers, network access points, base stations, and/or the like.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” “calculator,” “device,” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical data storage device, a magnetic data storage device, or any suitable combination of the foregoing. In the context of this document, a computer readable storage medium may be any tangible medium that can include, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the present invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks or modules.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks or modules.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks or modules.

It is to be appreciated that the term “processor” as used herein is intended to include any processing device, such as, for example, one that includes a CPU (central processing unit) and/or other processing circuitry. It is also to be understood that the term “processor” may refer to more than one processing device and that various elements associated with a processing device may be shared by other processing devices.

The term “memory” as used herein is intended to include memory associated with a processor or CPU, such as, for example, RAM, ROM, a fixed memory device (e.g., hard drive), a removable memory device (e.g., diskette), flash memory, etc. Such memory may be considered a computer readable storage medium.

In addition, the phrase “input/output devices” or “I/O devices” as used herein is intended to include, for example, one or more input devices (e.g., keyboard, mouse, scanner, etc.) for entering data to the processing unit, and/or one or more output devices (e.g., speaker, display, printer, etc.) for presenting results associated with the processing unit.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A method for making prognostic prediction scores during a pre-dialysis period on an incidence of events in future dialysis, the method comprising:
learning a meta-training model that simultaneously classifies dialysis in-distribution events; and
detecting out-of-distribution (OOD) events during model personalization by employing:
a data preprocessing component to extract different parts of data from historical medical records of patients to generate a meta-training dataset;
a meta-training component to analyze the meta-training dataset, the meta-training component including a class pool generator, a task generator, a prototype network, an attention component, and a model training component, the class pool generator splitting training classes into a first class pool for generating training tasks and a second class pool for generating a distribution statistics dictionary for transfer learning;
a storage component to store the meta-training model for distribution to local machines for further fine-tuning, personalization, and deployment; and
a personalization component including a local data collection component, and a class and OOD detector component, the class and OOD detector component using an energy score and a pre-defined threshold for estimating out-of-distribution samples.

2. The method of claim 1, wherein the data preprocessing component further removes noisy information and fills some missing values by using mean values of corresponding features in the historical medical records.

3. The method of claim 2, wherein the training tasks of the first class pool include a support set and a query set, the support set generated by only selecting training classes representing in-distribution data.

4. The method of claim 3, wherein the distribution statistics dictionary of the second class pool includes a mean and a variance of every class in the second class pool.

5. The method of claim 4, wherein the task generator includes:
a sampler for sampling several classes as the in-distribution data in the support set;
a sampler for sampling data in the in-distribution classes to constitute the query set; and
a sampler for randomly sampling several other classes as out-of-distribution data to constitute the query set.

6. The method of claim 1, wherein the prototype network of the meta-training component is a dual-channel combination network that encodes input data into feature vectors.

7. The method of claim 6, wherein the prototype network includes a static channel for processing static and low frequency temporal features and a temporal channel for processing high frequency temporal features.

8. A non-transitory computer-readable storage medium comprising a computer-readable program for making prognostic prediction scores during a pre-dialysis period on an incidence of events in future dialysis, wherein the computer-readable program when executed on a computer causes the computer to perform the steps of:
learning a meta-training model that simultaneously classifies dialysis in-distribution events; and
detecting out-of-distribution (OOD) events during model personalization by employing:
a data preprocessing component to extract different parts of data from historical medical records of patients to generate a meta-training dataset;
a meta-training component to analyze the meta-training dataset, the meta-training component including a class pool generator, a task generator, a prototype network, an attention component, and a model training component, the class pool generator splitting training classes into a first class pool for generating training tasks and a second class pool for generating a distribution statistics dictionary for transfer learning;
a storage component to store the meta-training model for distribution to local machines for further fine-tuning, personalization, and deployment; and
a personalization component including a local data collection component, and a class and OOD detector component, the class and OOD detector component using an energy score and a pre-defined threshold for estimating out-of-distribution samples.

9. The non-transitory computer-readable storage medium of claim 8, wherein the data preprocessing component further removes noisy information and fills some missing values by using mean values of corresponding features in the historical medical records.

10. The non-transitory computer-readable storage medium of claim 9, wherein the training tasks of the first class pool include a support set and a query set, the support set generated by only selecting training classes representing in-distribution data.

11. The non-transitory computer-readable storage medium of claim 10, wherein the distribution statistics dictionary of the second class pool includes a mean and a variance of every class in the second class pool.

12. The non-transitory computer-readable storage medium of claim 11, wherein the task generator includes:
a sampler for sampling several classes as the in-distribution data in the support set;
a sampler for sampling data in the in-distribution classes to constitute the query set; and
a sampler for randomly sampling several other classes as out-of-distribution data to constitute the query set.

13. The non-transitory computer-readable storage medium of claim 8, wherein the prototype network of the meta-training component is a dual-channel combination network that encodes input data into feature vectors.

14. The non-transitory computer-readable storage medium of claim 13, wherein the prototype network includes a static channel for processing static and low frequency temporal features and a temporal channel for processing high frequency temporal features.

15. A system for making prognostic prediction scores during a pre-dialysis period on an incidence of events in future dialysis, the system comprising:
a data preprocessing component to extract different parts of data from historical medical records of patients to generate a meta-training dataset;
a meta-training component to analyze the meta-training dataset, the meta-training component including a class pool generator, a task generator, a prototype network, an attention component, and a model training component, the class pool generator splitting training classes into a first class pool for generating training tasks and a second class pool for generating a distribution statistics dictionary for transfer learning;
a storage component to store a meta-training model for distribution to local machines for further fine-tuning, personalization, and deployment; and
a personalization component including a local data collection component, and a class and OOD detector component, the class and OOD detector component using an energy score and a pre-defined threshold for estimating out-of-distribution samples,
wherein the data preprocessing component, the meta-training component, the storage component, and the personalization component are collectively used to learn the meta-training model that simultaneously classifies dialysis in-distribution events and detects out-of-distribution (OOD) events during model personalization.

16. The system of claim 15, wherein the data preprocessing component further removes noisy information and fills some missing values by using mean values of corresponding features in the historical medical records.

17. The system of claim 16, wherein the training tasks of the first class pool include a support set and a query set, the support set generated by only selecting training classes representing in-distribution data.

18. The system of claim 17, wherein the distribution statistics dictionary of the second class pool includes a mean and a variance of every class in the second class pool.

19. The system of claim 18, wherein the task generator includes:
a sampler for sampling several classes as the in-distribution data in the support set;
a sampler for sampling data in the in-distribution classes to constitute the query set; and
a sampler for randomly sampling several other classes as out-of-distribution data to constitute the query set.

20. The system of claim 15,
wherein the prototype network of the meta-training component is a dual-channel combination network that encodes input data into feature vectors; and
wherein the prototype network includes a static channel for processing static and low frequency temporal features and a temporal channel for processing high frequency temporal features.
